(19) United States
(12) Patent Application Publication
(10) Pub. No.: US 2003/0175848 A1
(43) Pub. Date: Sep. 18, 2003

(54) Substituted dithiobisnitrobenzenes and their applications

(75) Inventors: Elisabeth Davioud-Charvet, Perenchies (FR); Christian Sergheraert, Morbecque (FR); Katja Becker-Brandenburg, Wetzlar (DE); Heiner Schirmer, Heidelberg (DE)

Correspondence Address: ALSTON & BIRD LLP, BANK OF AMERICA PLAZA, 101 SOUTH TRYON STREET, SUITE 4000, CHARLOTTE, NC, 28280-4000, US

(73) Assignee: Institut Pasteur De Lille

(21) Appl. No.: 10/348,678

(22) Filed: Jan. 22, 2003

Related U.S. Application Data
(63) Continuation of application No. 09/674,446, filed on Nov. 28, 2000, now abandoned.
Application No. 09/674,446 is a 371 of international application No. PCT/FR99/01215, filed on May 25, 1999.

(30) Foreign Application Priority Data
May 25, 1998 (FR) 98 06541

(51) Int. Cl. C12Q 1/26; C 07D 4 3/02; C 07D 4 1/02; C07C 323/33

(52) U.S. Cl. 435/25; 546/186; 548/517; 564/336; 568/22; 544/357

(57) ABSTRACT

The invention concerns substituted dithio-bis-nitrobenzenes (dithio-bis-nitrobenzylamines, dithio-bis-nitrobenzamides or dithio-bis-nitrophenacyls) and their uses as alternative substrates for NAD(P)H-dependent disulphide-reductase enzymes, such as trypanothion reductase and thioredoxine reductase and as a means for screening inhibitors of said enzymes. The invention also concerns an enzymatic test using said substrates and assay kits containing them. Said dithio-bis-nitrobenzenes correspond to general formula (I).

The present invention relates to substituted dithiobisnitrobenzenes (dithiobisnitrobenzylamines, dithiobisnitrobenzamides or dithiobisnitrophenacyls) and their applications as alternative substrates for NAD(P)H-dependent disulphide reductase enzymes such as trypanothione reductase, thioredoxin reductase or lipoamide dehydrogenase and as a means for screening inhibitors of these enzymes.

The present invention also relates to an enzymatic test using the said substrates as well as to an assay kit containing them.

Trypanothione reductase (TR) (A. H. Fairlamb et al., Annu. Rev. Microbiol., 1992, 46, 695-729), which is present in many parasites, more particularly in the Trypanosomatidae, catalyses the reduction of trypanothione disulphide (T(S)₂), a bis(glutathionyl)spermidine conjugate to the dithiol trypanothione (T(SH)₂). Since the parasites do not possess glutathione reductase, trypanothione is the main source of thiol groups for the parasites and allows a reducing intracellular medium to be maintained. Consequently, TR is a potential target for medicaments intended for combating diseases caused by a trypanosome (sleeping sickness, Chagas' disease, for example).

The article in the name of A. H. Fairlamb et al. cited above describes the structural characteristics of TR. It teaches in particular the presence of a hydrophobic region and of negatively charged residues at the level of the active site of the TR enzyme and therefore recommends, as possible alternative substrates for trypanothione disulphide, hydrophobic peptide compounds possessing a positively charged secondary or tertiary amine function.

Other documents also describe analogues of the natural substrate for trypanothione reductase (A. El-Waer et al., Anal. Biochem, 1991, 198, 212-216; A. El-Waer et al., Int. J. Peptide Protein Res., 1993, 41, 141-146; R. Jaouhari et al., Amino Acids, 1995, 9, 327-342; R. Jaouhari et al., Amino Acids, 1995, 9, 343-351; I. R. Marsh et al., Eur. J. Biochem., 1997, 243, 690-694).

The analogues described in these various documents are analogues of the physiological substrate of the enzyme, oxidized N¹, N⁸-bis(glutathionyl)-spermidine or trypanothione disulphide. They all possess a peptide structure.

These analogues differ from the physiological substrate in the replacement of its spermidine part by 3-dimethylaminopropylamine (DMAPA) groups (A. El-Waer et al., Anal. Biochem, 1991, 198, 212-216; A. El-Waer et al., Int. J. Peptide Protein Res., 1993, 41, 141-146; R. Jaouhari et al., Amino Acids, 1995, 9, 327-342; R. Jaouhari et al., Amino Acids, 1995, 9, 343-351) or in the chemical modification of the glutamic acid side chains (I. R. Marsh et al., Eur. J. Biochem., 1997, 243, 690-694).

These documents show that such analogues are less efficient than the natural substrate (A. El-Waer et al., Anal. Biochem, 1991, 198, 212-216, for example).

Indeed, the dynamic specificities of these alternative substrates (K_{cat}/Kₘ) towards TR are significantly less than those of the natural substrate.

Thioredoxin reductase (TrxR) catalyses the NADPH-dependent reduction of thioredoxin (Trx); the human and murine TrxRs are selenoenzymes. The reduced Trx participates in the redox regulation of many enzymes which are essential for cell life. Furthermore, outside the cell, reduced Trx plays the role of activator of cell growth (autocrine factor). The key role played by reduced Trx makes it possible to think that the inhibition of the enzyme responsible for its reduction will result in cell growth being stopped.

In humans, it has been observed that the activity of this enzyme is significantly increased (by at least a factor of 10) in cancer cells: the inhibition of thioredoxin reductase should therefore make it possible to treat diseases in which rapid cell multiplication exists (cancer, malaria, psoriasis, autoimmune diseases, parasitic diseases).

DNTB has been proposed as replacement substrate, for measuring the activity of thioredoxin reductase; however, the Kₘ values for DNTB for the TrxRs are high and incompatible in the context of a kinetic analysis of TrxR.

Patent U.S. Pat. No. 4,975,367 describes the use of DTNB or of some of its analogues as thiol indicator in a catalytic system comprising

i) a disulphide reductase,

ii) a disulphide substrate, namely the natural substrate for the enzyme used, and

iii) the said thiol indicator,

the disulphide reductase catalysing the reaction between NAD(P)H, formed during previous catalytic reactions, and the disulphide substrate, so as to produce a compound which can interact with the thiol indicator to produce a change in colour. In this patent, the disulphide reductase reacts with its natural substrate, and then DTNB or one of its analogues, called “thiol indicator”, reacts with the thiol thus formed, producing a coloured compound.

To develop medicaments capable of inhibiting these various enzymes, it is necessary to have alternative substrates whose cost is reasonable and which are as effective as the natural substrates (Kₘ, K_{cat} and K_{cat}/Kₘ, of comparable values) and which induce the formation of a substance which can be directly measured by spectrometry or fluorometry during the enzymatic reaction, so as to have a suitable tool, allowing automation, for the screening of substances inhibiting the said enzymes.

However, all the substrates currently available do not make it possible to have an effective tool for screening inhibitors of these enzymes.

Accordingly, the inventors set themselves the aim of developing novel substrates, which are more suitable for the requirements of practical use than the prior art substrates, in particular:

in that they are not very expensive,

in that they are capable of serving as substrates for all the NAD(P)H-dependent disulphide reductases,

in that they release a chromophore or a fluorophore during the enzymatic catalysis, which can be easily measured by visible spectrometry or by fluorometry, and

in that they have properties which are more similar to those of the natural substrate than the substitute substrates previously described, in particular both as regards trypanothione reductase and thioredoxin reductase.

The subject of the present invention is substituted dithiobisnitrobenzenes whose general formula I is the following:

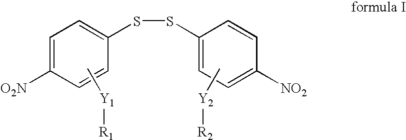

in which:

R₁ and R₂, which may be identical or different, represent:

a group NR₃R₄, in which R₃ and R₄, which may be identical or different, represent a hydrogen atom or a linear, branched or cyclic C₁-C₂₀ carbon chain comprising 1, 2 or 3 protonable nitrogen atoms,

a group OR₅, in which R₅ represents a hydrogen atom or an optionally substituted, linear, branched or cyclic C₁-C₂₀ carbon chain comprising 1, 2 or 3 protonable nitrogen atoms, on the condition that R₁ or R₂ represents a group NR₃R₄, in which R₃ and R₄, which may be identical or different, represent a hydrogen atom or a linear, branched or cyclic C₁-C₂₀ carbon chain comprising 1, 2 or 3 protonable nitrogen atoms,

an optionally substituted, linear, branched or cyclic C₁-C₂₀ carbon chain comprising 1 to 4 protonable nitrogen atoms,

or alternatively R₁ and R₂ are covalently linked, to form a macroring by formation of a chain of the NH—R₆—NH type in which R₆ represents a C₁-C₂₀ carbon chain comprising 1 to 4 protonable nitrogen atoms,

Y₁ and Y₂, which may be identical or different, represent a CH₂ group or a CO group, on the condition that when Y₁ and/or Y₂ represent a CH₂ group, R₁ and R₂, which may be identical or different, represent solely a group NR₃R₄, in which R₃ and R₄, which may be identical or different, represent a hydrogen atom or a linear, branched or cyclic C₁-C₂₀ carbon chain comprising 1, 2 or 3 protonable nitrogen atoms,

and on the condition that, when Y₁ and Y₂ represent a CO group, R₁ and R₂ do not simultaneously represent either a group —NH—(CH₂)₃—N(CH₃)₂ or a group:

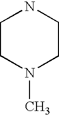

The said substituted dithiobisnitrobenzenes can in particular be used as alternative substrates for NAD(P)H-dependent disulphide reductases such as the enzymes trypanothione reductase (TR), for example trypanothione reductase from Trypanosomia cruzi or from Leishmania and thioredoxin reductase (TrxR), for example human thioredoxin reductase (hTrxR) or from Plasmodium falciparum (PfTrxR) or lipoamide dehydrogenase.

According to an advantageous embodiment of the said derivatives, Y₁ and Y₂ are identical and represent a CO group; such compounds represent either dithiobisnitrobenzamides or dithiobisnitrophenacyls and correspond to the following formula II:

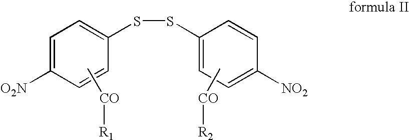

in which R₁ and R₂ have the same meaning as above.

According to an advantageous arrangement of this embodiment, R₁ and R₂ are identical and represent a group NR₃R₄, in which R₃ and R₄ are different: one represents a hydrogen atom and the other represents a group

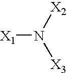

in which the groups X₁, X₂ and X₃ represent an optionally substituted, linear, branched or cyclic C₁-C₂₀ carbon chain comprising 1 to 4 protonable nitrogen atoms, an alkylamine group or an arylamine group, which are optionally substituted or branched, or alternatively X₁ and X₂ form a C₄-C₈ cyclic carbon chain which may optionally contain at least one nitrogen atom and X₃ represents a C₁-C₈ alkyl group, an alkylamine group or an arylamine group, which are optionally substituted or branched, the said group

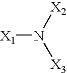

being linked by a carbon atom of at least one of the groups X₁, X₂ or X₃ to the nitrogen atom of the group NR₃R₄; R₃ or R₄ represent for example the following group:

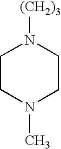

(compound 2); such compounds are dithiobisnitrobenzamides.

According to another advantageous arrangement of this embodiment, R₁ and R₂ are different, one representing a group OR₅ and the other representing a group NR3R₄, as defined above. Compound 4 represented below is for example obtained.

According to yet another advantageous arrangement of this embodiment, R₁ and R₂ form a chain NH—R₆—NH in which R₆ represents a C₁-C₂₀ carbon chain comprising 1 to 4 protonable nitrogen atoms; compound 5 represented below is thus obtained.

These various dithiobisnitrobenzamides preferably exhibit the following structural characteristics:

two phenyl nuclei are linked by a disulphide bridge,

the two aromatic rings are substituted with a nitro group at the para position,

at least one of the aromatic rings is substituted, at the ortho or meta position, with a group CO—R₁ or CO—R₂, with the following different variants:

the two aromatic rings are substituted, either at the ortho position, or at the meta position, with an amide group CO—NR₃R₄, as defined above (compound 2),

one of the rings is substituted with an amide group CO—NR₃R₄, as defined above (at the ortho or meta position), the other ring being substituted with a carboxyl or ester group (at the ortho or meta position) (compound 4),

the two aromatic rings are linked by a cyclic benzamide, at the ortho or meta position (compounds 5).

The structural formulae of the compounds 2, 4 and 5 are the following:

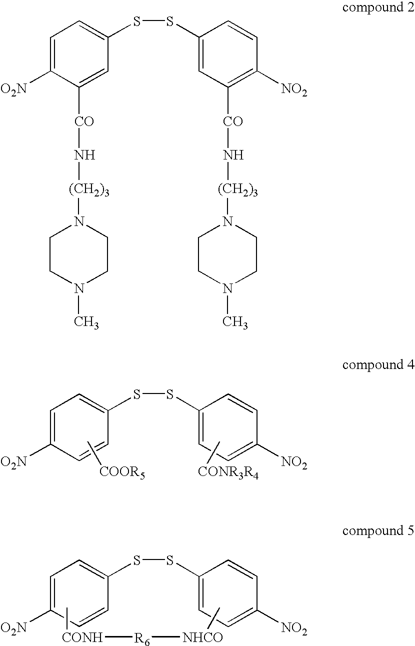

According to yet another advantageous arrangement of this embodiment, R₁ and R₂, which may be identical or different, represent an optionally substituted, linear, branched or cyclic C₁-C₂₀ carbon chain comprising 1, 2, 3 or 4 protonable nitrogen atoms; dithiobisnitrophenacyls are then obtained.

According to another advantageous embodiment of the said derivatives, Y₁ and Y₂ are identical and represent a CH₂ group; such compounds are dithiobisnitrobenzylamines and correspond to the following formula III:

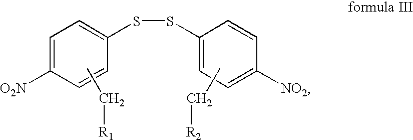

in which R₁ and R₂, which may be identical or different, represent a group NR₃R₄ in which R₃ and R₄, which may be identical or different, represent a hydrogen atom or an optionally substituted, linear, branched or cyclic C₁-C₂₀ carbon chain comprising 1, 2 or 3 protonable nitrogen atoms.

Optionally substituted, linear, branched or cyclic carbon chain is understood to mean, for the purposes of the present invention, a C₁-C₂₀ alkyl, aryl, alkoxyl, aralkyl, cycloalkyl, alkylamine or arylamine group (without counting the additional optional substituents).

More precisely:

the expression alkyl denotes linear or branched or optionally substituted C₁-C₆ alkyl groups, such as for example methyl, ethyl, propyl, butyl, isopropyl, isobutyl, sec-butyl or tert-butyl groups. These groups form with one or more nitrogen atoms alkylamino groups such as: dimethylamino, diethylamino, butylamino, diisopropylamino, di-tert-butylamino, methyl-tert-butylamino, propyl-tert-butylamino, methylpropylamino, methylethylamino;

the expression alkoxyl denotes C₁-C₆ alkoxyl groups such as the methoxyl, ethoxyl, propoxyl, butoxyl, isopropoxyl, isobutoxyl, sec-butoxyl and tert-butyloxyl groups;

the expression aryl denotes for example a phenyl radical optionally substituted with one or more alkyl or alkoxyl radicals or a 4-, 5- or 6-membered heterocyclic aromatic radical containing 1 to 4 heteroatoms of nitrogen;

the expression aralkyl denotes the groups of general formula Ar—CH₂, in which Ar represents an aryl group;

the expression cycloalkyl may denote for example a cyclopropyl, cyclobutyl, cyclopentyl or cyclohexyl radical;

the expression nitrogen-containing C₄-C₈ heterocycle denotes for example the piperidine, pyrrolidine, piperazine, imidazole and indoline radicals and the like;

the various substituents are generally alkyl groups as defined above.

The subject of the present invention is also a method of preparing the dithiobisnitrobenzamides, corresponding to the products of formula I, in which:

Y₁ and Y₂ are identical and each represent a CO group, and

R₁ and R₂, which may be identical or different, represent a group NR₃R₄, which method is characterized in that it comprises:

(1) the conversion of dithiobisnitrobenzoic acid (DTNB) (5,5′-dithiobis(2-nitrobenzoic) acid or its isomer 6,6′-dithiobis(3-nitrobenzoic) acid) to an acylated derivative, in the presence of at least one coupling agent,

(2) the formation of an amide, from the product obtained in (1), by the action of an amine present in excess, and

(3) the purification of the product obtained in (2).

In accordance with the invention, the coupling agents are selected from the coupling agents described in Methods in Enzymology, 1997, 289, 104-126 (F. Albericio and L. A. Carpino; published by Fields G. B., Academic Press), for example the coupling agents HOBt ((N-hydroxybenzotriazole) or HBTU (2-(1H-benzotriazol-1-yl)-1,1,3,3-tetramethyluronium).

Also in accordance with the invention, step (1) is carried out in the presence of a base.

The subject of the present invention is also a method of preparing the dithiobisnitrophenacyls, corresponding to the products of formula I, in which:

Y₁ and Y₂ are identical and each represent a CO group, and

R₁ and R₂, which may be identical or different, represent an optionally substituted, linear, branched or cyclic C₁-C₂₀ carbon chain comprising 1, 2, 3 or 4 protonable nitrogen atoms, which method is characterized in that it comprises:

(1) the formation of a diazoketone, from 5-chloro-2-nitrobenzoic or 2-chloro-5-nitrobenzoic acid,

(2) the hydrolysis of the diazoketone with hydrobromic acid,

(3) the substitution of the α-bromoketone with an amine and

(4) the formation of the disulphide, from the compound obtained in (3), by the action of Na₂S, S₈, in alcoholic medium, in accordance with scheme 1 below:

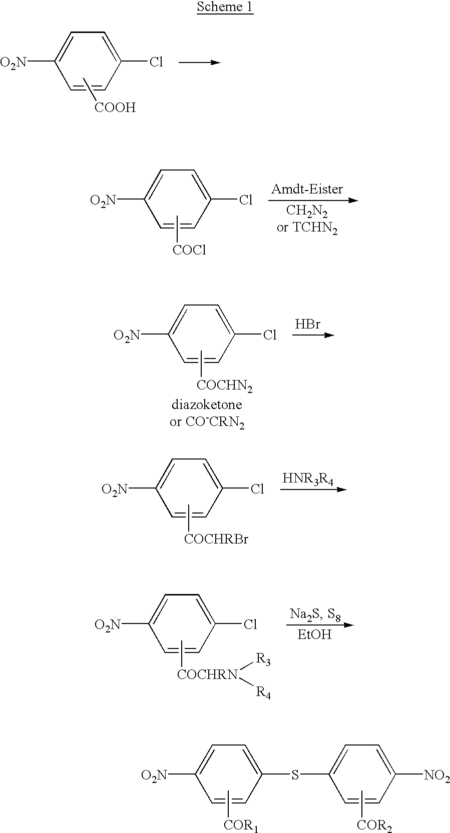

The Arndt-Eistert step is in particular described in Angew. Chem. Int., 1975, 14, 32-43 and the step for forming the disulphide is in particular described in J M Domagala et al., (Biorg. Med. Chem., 1997, 5, 3, 569-579).

The subject of the present invention is also a method of preparing the dithiobisnitrobenzylamines, corresponding to the products of formula I, in which:

Y₁ and Y₂ are identical and each represent a CH₂ group, and

R₁ and R₂, which may be identical or different, represent a group NR₃R₄, in which R₃ and R₄, which may be identical or different, represent a hydrogen atom or an optionally substituted, linear, branched or cyclic C₁-C₂₀ carbon chain comprising 1, 2 or 3 protonable nitrogen atoms, which method is characterized in that it comprises:

(1) the reductive amination of an aldehyde 5-chloro-2-nitrobenzaldehyde or 2-chloro-5-nitrobenzaldehyde; such a reaction is in particular described in J A Sclafani et al., J. Org. Chem., 1996, 61, 3221-3222, and

(2) the formation of the disulphide, from the compound obtained in (1), by the action of Na₂S, S₈, in alcoholic medium, under the same conditions as those stated above, in accordance with scheme 2 below:

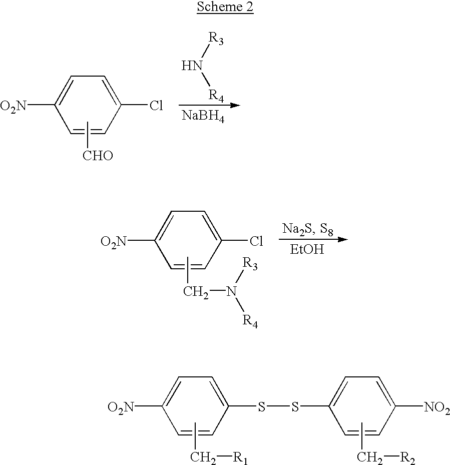

As a variant, the said products may be prepared from the following thiophenol: 2-mercapto-5-nitrobenzaldehyde, of the following formula:

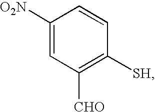

by the same reductive amination reaction as that described above, after protection of the thiophenol.

The subject of the present invention is also a method of preparing the dithiobisnitrobenzamides according to the invention, in which R₁ and R₂ are different, one representing a group OR₅ and the other representing a group NR₃R₄, R₃, R₄ and R₅ being as defined above, characterized in that it comprises the following steps:

(1) the conversion of 5,5′-dithiobis(2-nitrobenzoic) acid to a monoester, according to a self-catalysed Fisher esterification reaction, with the aid of an alcohol of general formula R₅OH, R₅ being as defined above,

(2) the separation of the monoester obtained in (1) from the 5,5′-dithiobis(2-nitrobenzoic) acid and the diester also formed in (1), by chromatography, and

(3) the formation of an amide at the level of the monoacid function of the compound isolated in (2), by the action of an amine of general formula NHR₃R₄ in excess, R₃ and R₄ being as defined above, in the presence of at least one coupling agent.

The Fisher reaction is as described by Tsang et al. in J. Am. Chem. Soc., 1994, 116, 3988-4005.

The coupling agents are as described above, for example HOBt and/or HBTU.

By way of example, the method of preparing the dithiobisnitrobenzamides according to the invention, in which R₁ and R₂ are different, one representing a group OR₅ and the other representing a group NR₃R₄, R₃, R₄ and R₅ being as defined above, comprises the steps summarized in the following scheme 3:

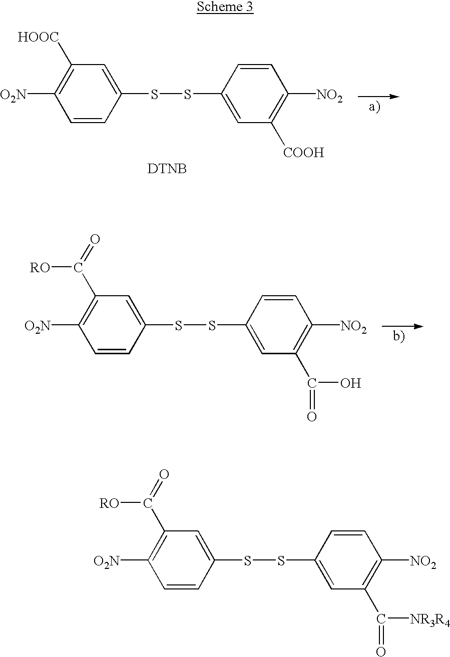

in which:

step a): alcohol R₅OH, reflux, 50 h,

step b): amine NHR₃R₄ (1.6 eq.), diisopropylethylamine (4 eq.), HOBt (1.25 eq.), HBTU (1.25 eq.) in dichloromethane, at room temperature.

The subject of the present invention is also a method of measuring the activity of the NAD(P)H-dependent disulphide reductases, whose active site comprises negatively charged amino acids and aromatic amino acids, capable of developing interactions with their substrate, either of the ionic type, or of the cation-Π type (enzymatic test), which method is characterized in that it comprises:

bringing the said enzymes into contact with a suitable substrate selected from the substituted dithiobisnitrobenzenes as defined above and the substituted dithiobisnitrobenzenes of formula I as represented above, in which Y₁ and Y₂ represent a CO group and R₁ and R₂ represent simultaneously a group —NH—(CH₂)₃—N(CH₃)₂ or a group:

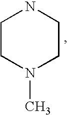

and

the direct detection of the thiolates formed, in particular by visible spectrophotometry.

When the substituted dithiobisnitrobenzenes are such that Y₁ and Y₂ represent a CO group and R₁ and R₂ represent simultaneously a group —NH—(CH₂)₃—N(CH₃)₂ or a group:

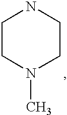

they correspond to the following compounds 1 and 3:

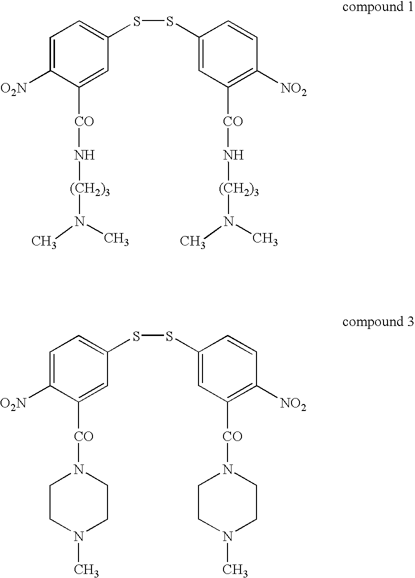

In accordance with the invention, the said enzymes are in particular selected from the group consisting of trypanothione reductase, thioredoxin reductase and lipoamide dehydrogenase.

The thiolates which are formed under the action of the said reductases, from the disulphide substrates according to the invention, in accordance with the following scheme:

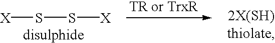

are directly detectable by visible spectrophotometry, because they have a chromophore group, which has an advantage compared with the enzymatic test described in M. Aumercier et al., Anal. Biochem., 1994, 223, 161-164, which describes an enzymatic test for evaluating the activity of the TR enzyme, of which the principle is to measure the quantity of residual disulphide present in the medium at the end of the enzymatic reaction. The measurement is carried out by visible spectrophotometry at 405 nm and requires derivatizing the disulphide with the CMTQ reagent (salt of 4-chloro-1-methyl-7-trifluoromethylquinoline), in order to make it detectable in the visible region. The thiols formed during the enzymatic reaction are made nonreactive beforehand with respect to the CMTQ, by alkylation with 2-vinylpyridine.

The subject of the present invention is also a kit or box for measuring the activity of the NAD(P)H-dependent disulphide reductases, as defined above, characterized in that it comprises, as substrate for the said enzymes, a substituted dithiobisnitrobenzene selected from the group consisting of the substituted dithiobisnitrobenzenes as defined above and the substituted dithiobisnitrobenzenes of formula I as represented above in which Y₁ and Y₂ represent a CO group and R₁ and R₂ represent simultaneously a group —NH—(CH₂)₃—N(CH₃)₂ or a group:

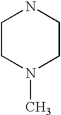

The subject of the present invention is also a method of screening and selecting products inhibiting an NAD(P)H-dependent disulphide reductase, characterized in that it comprises:

bringing a potential inhibitor into contact, in the presence of an NAD(P)H-dependent disulphide reductase, with a compound selected from the group consisting of the substituted dithiobisnitrobenzenes as defined above and the substituted dithiobisnitrobenzenes of formula I as represented above, in which Y₁ and Y₂ represent a CO group and R₁ and R₂ represent simultaneously a group —NH—(CH₂)₃—N(CH₃)₂ or a group:

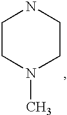

and

the calorimetric detection of the thiolates which may be formed.

The enzymatic test, as defined above, makes it possible to measure, by means of the thiolates formed, the activity of the NAD(P)H-dependent disulphide reductases such as the TR and TrxR enzymes and therefore to assess the capacity of various molecules to inhibit these reductases, in the presence of the various substituted dithiobisnitrobenzenes according to the invention as alternative substrates. Indeed, the Kₘ values observed with the substituted dithiobisnitrobenzenes and in particular the dithiobisnitrobenzenes according to the invention are particularly well suited to the kinetic analysis of TR and of TrxR and increase the sensitivity of the reaction to the inhibitors.

The subject of the present invention is also a method of assaying the NAD(P)H-dependent disulphide reductases, in a biological sample, characterized in that it comprises:

bringing the said biological sample, optionally treated, into contact with a suitable substrate selected from the group consisting of the substituted dithiobisnitrobenzenes as defined above and the substituted dithiobisnitrobenzenes of formula I as represented above, in which Y₁ and Y₂ represent a CO group and R₁ and R₂ represent simultaneously a group —NH—(CH₂)₃—N(CH₃)₂ or a group:

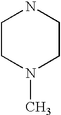

and

the direct detection of-the thiolates formed, in particular by visible spectrophotometry.

The subject of the present invention is in addition the use of the method of detection as defined above, for the diagnosis of pathologies where the activity of the NAD(P)H-dependent disulphide reductases is significantly increased (pathologies in which an increased cell proliferation is encountered, such as cancer for example).

Advantageously, in the processes and methods described above, the said NAD(P)H-dependent disulphide reductases are selected from the group consisting of trypanothione reductase, thioredoxin reductase and lipoamide dehydrogenase.

In addition to the preceding arrangements, the invention also comprises other arrangements, which will emerge from the description which follows, which refers to exemplary embodiments of the method which is the subject of the present invention as well as to the accompanying drawings, in which:

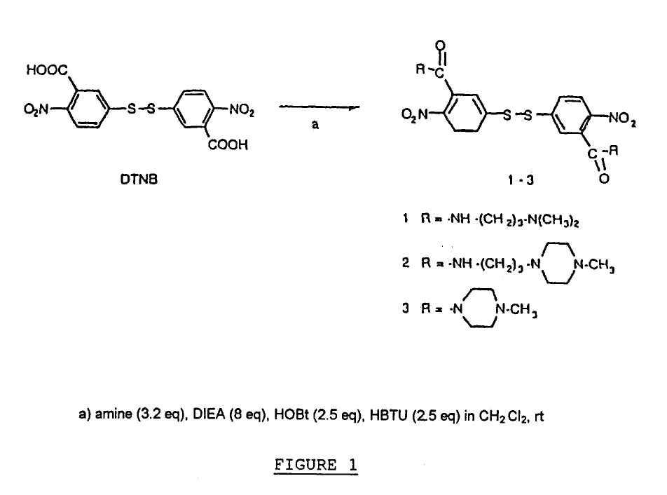

FIG. 1 illustrates the synthesis of compounds 1-3;

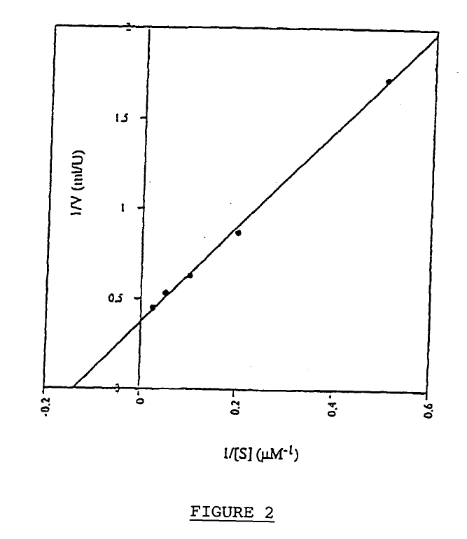

FIG. 2 corresponds to a Lineweaver-Burk curve for the reduction of compound 1 with hTrxR; the Kₘ, value is 7 μM; this figure comprises on the x-axis the reciprocal of the concentrations of substrate 1/[S] in μM⁻¹ and on the y-axis the reciprocal of the rate 1/V in ml/U;

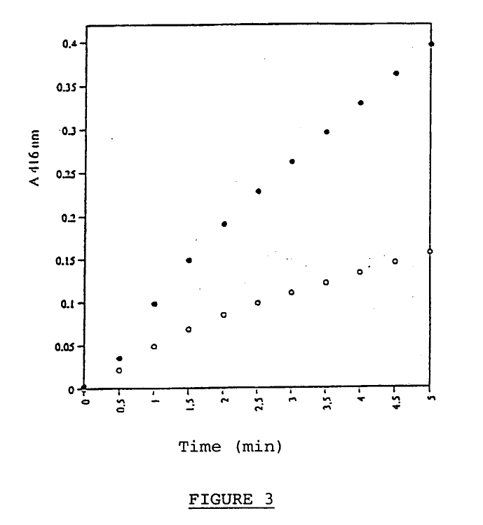

FIG. 3 represents the formation of 5-thio-2-nitrobenzamide catalysed by TcTR and measured by spectrophotometry at 416 nm, as a function of time, in the absence (•)or in the presence (O) of 50 μM of clomipramine;

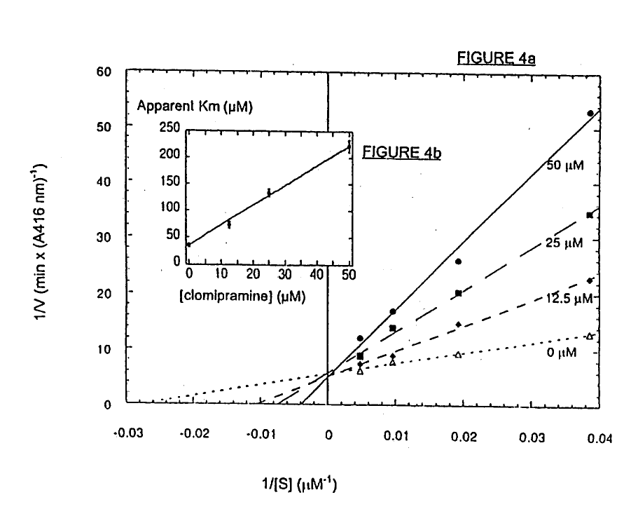

FIG. 4a corresponds to a Lineweaver-Burk curve for the reduction of compound 1 with TrR; this figure comprises on the x-axis the reciprocal of the concentrations of substrate 1/[S] in μM⁻¹ and on the y-axis the reciprocal of the rate 1/V in min×(A416 nm)⁻¹ (with min=minutes; A416 nm=absorbance measured at 416 nm). FIG. 4b illustrates the curve for the apparent Kₘ values (on the y-axis, in μM) for TR, as a function of the concentration of clomipramine (on the x-axis, in μM) [I], deduced from the Lineweaver-Burk curve represented in FIG. 4a, with compound 1 as substrate;

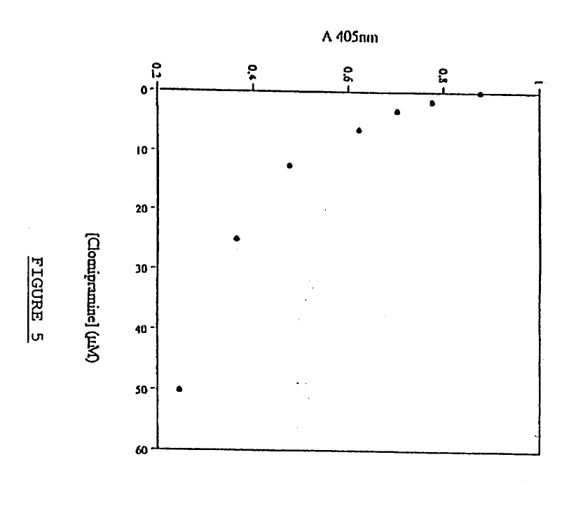

FIG. 5 illustrates the formation of 5-thio-2-nitrobenzamide catalysed by TCTR and measured by the absorbance at 405 nm as a function of the concentrations of inhibitor in the presence of compound 1 as substrate.

It should be clearly understood, however, that these examples are given solely by way of illustration of the subject of the invention and do not constitute in any manner a limitation thereto.

### EXAMPLE 1:

### Method of Preparing Compounds 1, 2 and 3

Compounds 1-3 are prepared from the commercially available 5,5′-dithiobis(2-nitrobenzoic) acid (DTNB). The general method of synthesis allows the preparation of benzamides in large quantities (on the gram scale). In each case, the amine is used in excess (3.2 eq.) in the inert solvent CH₂Cl₂ with the addition of a cobase, DIEA (N,N-diisopropylethylamine) and coupling agents, HOBt and HBTU, in order to convert benzoic acid to an active acylating species. The disulphides obtained, which are acylated with a side chain of the polyamine type, are stored in the hydrochloride form (compound 1) or oxalate form (compounds 2 and 3).

The general procedure for the synthesis of the 5,5′-dithiobis(2-nitrobenzamides) 1-3 is more particularly illustrated below by the preparation of compound 1 (also see FIG. 1).

1.94 g (2.5 eq.) of HOBt, 4.78 g (2.5 eq.) of HBTU and 7.02 ml (8 eq.) of DIEA are added to a solution of 2 g (5.05 mmol) of DTNB in 28 ml of CH₂Cl₂. The mixture is stirred at 4° C. for 15 minutes.

An amine, N,N-dimethylpropylamine, (3.2 eq.) is added at 0° C. and the stirring of the mixture is continued for 20 minutes at 0° C. The reaction mixture is heated up to room temperature and maintained at this temperature for one hour. The solution of CH₂Cl₂ is optionally diluted with 160 ml of CH₂Cl₂, and then washed with water, dried over MgSO₄ and evaporated.

A purification by thin-layer chromatography (alumina gel; solvent 1: CH₂Cl₂—MeOH (95-5) and solvent 2: CH₂Cl₂—MeOH (80-20)) makes it possible to obtain 900 mg of 5,5′-dithiobis(2-nitrobenzamide) 1 in the form of a yellow oil; yield 32%; R_{f} 0.42 in CH₂Cl₂—MeOH (95-5).

The 5,5′-dithiobis(2-nitrobenzamides) 2 and 3 are also isolated in the form of a yellow oil; 2: yield 12%, R_{f} in CH₂Cl₂—MeOH (95-5): 3; yield 13%; R_{f} 0.6 in CH₂Cl₂—MeOH (82.5-5).

The hydrochloride form of product 1 is obtained by dissolving the base in 18 ml of MeOH, followed by the addition of 376 μl of Me₃SiCl (2 eq.).

The reaction mixture is stirred at room temperature for 5 minutes and the evaporation of the reagents leads to a light yellow amorphous powder: melting point: 113-114° C.; TOF-PDMS: 564.4 (M⁺), 282.7; ¹H NMR (300 MHz) δ1.80 (qt, J=6.0 Hz, 4H, CH₂—CH₂—CH₂), 2.25 (s, 12 H, N(CH₃)₂), 2.55 (t, J=6.0 Hz, 4H, CH₂—N—(CH₃)₂, 3.50 (m, 4H, CH₂—NH), 7.55 (d, Jₘₑₜₐ=2.0 Hz, 2H, H₆), 7.65 (dd, Jₒᵣₜₕₒ=8.5 Hz, Jₘₑₜₐ=2.0 Hz, 2H, H₄), 8.00 (d, Jₒᵣₜₕₒ=8.5 Hz, 2H, H₃), 8.10 (bs, 2H, NHCO).

The oxalates are obtained by adding dropwise a saturated solution of oxalic acid in AcOEt (ethyl acetate) to the saturated solution of the amine-containing derivatives 2-3.

The mixture is maintained at 4° C. for 3 hours; the salts are obtained in the form of light yellow amorphous powders after filtration and successive washes with ice, cold AcOEt and ether.

2 (Oxalate): melting point: 122-123° C.; TOF-PDMS =674.9 (M⁺), 336.6; ¹H NMR (300 MHz, CD₃SO, 340° K) δ1.70 (qt, J=7.0 Hz, 4H, CH₂—CH₂—CH₂), 2.10 (s, 6H, NCH₃), 2.6 (m, 4H, CH₂—CH₂—N), 3.30 (m, 4H, CH₂—NHCO), 2.70-3.70 (m, 16H, N—CH₂—CH₂—N), 7.75 (d, Jₘₑₜₐ=2.0 Hz, 2H, H₆), 7.80 (dd, 2H, Jₒᵣₜₕₒ=8.5 Hz, Jₘₑₜₐ=2.0 Hz, H₄), 8.10 (d, Jₒᵣₜₕₒ=8.5 Hz, 2H, H₃), 8.60 (t, J=5.6 Hz, 2H, NHCO).

3 (Oxalate): melting point: 182-183° C.; TOF-PDMS: 562.1 (M⁺), 281.4; ¹H NMR (300 MHz, CD₃SO, 340° K) δ2.10 (s, 6H, N—CH₃), 2.60-3.80 (m, 16H, CH₂—CH₂—N), 7.70 (d, Jₘₑₜₐ=2.0 Hz, 2H, H₆), 7.8 (dd, Jₒᵣₜₕₒ=8.5 Hz, Jₘₑₜₐ=2.0 Hz, 2H, H₄), 8.2 (d, Jₒᵣₜₕₒ=8.5 Hz, 2H, H₃).

All the melting points were determined with the aid of a Büchi apparatus and are not corrected.

All the reactions were analysed by thin-layer chromatography (TLC) (CH₂Cl₂—MeOH, 95-5) carried out on alumina gel plates (0.2 mm) (Macherey-Nagel Polygram alox N/UV₂₅₄), using UV light as visualizing agent or a Reindel-Hoppe solution as developing agent.

The ¹H spectrum is obtained with a Brucker 300 MHz spectrometer; the mass spectrum is recorded on a desorption spectrometer (PDMS) (source: californium).

### EXAMPLE 2

### Measurement of the Activity of the Trypanothione Reductase and Thioredoxin Reductase Enzymes, with, as Substrates, Compounds 1, 2 and 3 according to Example 1

Materials and Methods

Enzymes

The trypanothione reductase from Trypanosoma cruzi (TcTR) is isolated from the Escherichia coli SG5 strain carrying a vector for overexpression of trypanothione reductase (28), pIBITczTR.

The TcTR concentration is determined by measuring the number of subunits containing FAD at 461 nm (ε=11.3 mM⁻¹×cm⁻¹); the enzymatic activity is tested as specified in (28). One TR unit corresponds to 1 μmol of T(S)₂ reduced per minute at 25° C. in a buffer A (20 mM Hepes, pH 7.25 containing 1 mM EDTA and 0.15 M KCl).

The enzyme stock solutions used for the kinetic determinations are pure (verification in SDS-PAGE) and have a specific activity of 137 U/mg in the T(S)₂ reduction test performed in the presence of 500 μM NADPH and 518 μM T(S)₂ in a buffer A.

The human thioredoxin reductase (hTrxR) is purified from placenta as specified in J. E. Oblong et al., Biochemistry, 1993, 32, 7271-7277.

The recombinant thioredoxin reductase from Plasmodium falciparum (PfTrxR) is expressed in the E. coli strain deficient in glutathione reductase SG5 and purified according to a procedure similar to that used for the recombinant human glutathione reductase (26, 30).

The enzymatic activities of these enzymes are usually determined with the aid of a test of reduction of DTNB, as follows:

The enzyme is added to a reaction mixture comprising 100 mM potassium phosphate, 2 mM EDTA, pH 7.4 and 3 mM DTNB; after the addition of 200 μM NADPH, the increase in the absorbence is measured at 412 nm and at 25° C. Using the DTNB test, one TrxR unit is defined as the NADPH-dependent production of 2 μmol of 5-thio-2-nitrobenzoate (ε_{412 nm}=13.6 mM⁻¹.cm⁻¹). The TrxR concentrations are determined by measuring subunits containing FAD at 450 nm (ε=11.3 mM⁻¹.cm⁻¹). The enzyme stock solutions, used for the kinetic determinations, are pure (verification in SDS-PAGE) and have a specific activity of 31 u/mg (hTrxR) and of 5.9 U/mg (PfTrxR), respectively, in the DTNB test.

Conditions for the Kinetic Studies

Before use, the substrates (compounds 1, 2 and 3) are dissolved in DMSO; precise concentrations (10 mM in the stock solution) are either calculated spectrophotometrically in 20 mM HEPES, 1 mM EDTA, 150 mM KCl, pH 7.25 (buffer A) based on the respective thiolate concentrations after enzymatic reduction, or obtained by measuring the absorbence at λmax=327 nm, using the molar extinction coefficient (ε_{327 nm}=15,533 M⁻¹×cm⁻¹ for compound 1, for example).

All the kinetic studies are carried out in the same buffer at 25° C., in the presence of 200 μM of NADPH.

Colorimetric Test

Measurement of the TR Activity

The initial concentration of compound 1 (in its disulphide form) is determined, at λmax=327 nm, using its molar extinction coefficient ε_{327 nm}=15,533 M⁻¹×cm⁻¹.

The measurements are carried out in microtitre plates. 80 μl of an enzymatic solution containing 28×10⁻⁴ U of TcTR are added to 10 μl of a solution of substrate in buffer A containing in addition Me₂SO comprising 30 nmol of compound 1, 2 or 3 and 50 nmol of NADPH (final concentrations: 300 μM of compound 1, 2 or 3 and 500 μM of NADPH); the NADPH is used in a very large excess because it is destroyed in solution and the solution should remain stable throughout the day (in order to carry out the screening tests). These conditions are those of the microplate test in the presence or in the absence of inhibitors and are different from the spectrophotometric test conditions in which the Kₘ values are measured.

Positive and negative controls are also prepared.

Measurement of the TrxR Activity

It is carried out under the same conditions as above; it comprises however the following modifications:

all the reactions in the presence of TrxR are carried out in buffer C (100 mM sodium phosphate and 2 mM EDTA, pH 7).

the final concentration of compound 1, 2 or 3 is 200 μM. The reaction is triggered by the addition of 80 μl of an enzymatic solution containing either 8×10⁻⁴ U of native hTrxR or 32×10⁻⁴ U of recombinant PfTrxR.

Expression of the Results

The enzymatic activity is assessed by the formation of the thiolates produced as specified above.

The calorimetric detection of the thiolates formed is immediate because 5-thio-2-nitrobenzamide is a yellow chromophoric group. The presence of 5-thio-2-nitrobenzamide and the disappearance of the starting disulphide in the reaction medium, after the complete enzymatic reaction, is demonstrated by a TOF-PDMS (Time Of Flight-Plasma Desorption Mass Spectrometry) analysis. For the three compounds 1-3, the absorption spectra of the thiolates formed were recorded by scanning wavelengths (190-600 nm). The values of λmax were determined at 416 nm for the three compounds, thus providing the molar absorption coefficients of 12188 (1), 10277 (2) and 10203 (3) M⁻¹×cm⁻¹ at 416 nm, in buffer A. By comparison with the absorbance values for the thiolates, the molar absorption coefficients of the starting disulphides are so low that they are almost negligible (>400 M⁻¹×cm⁻¹ at 416 nm). Since the majority of the microplate readers have a filter at 405 nm, the formation of the thiolates was measured at 405 nm, the molar absorption coefficients being very close (differences <6%) to the two wavelengths (416 and 405 nm). In the microtitre plate test, a maximum absorbance/absorbance of the background noise ratio of 13 is observed at 405 nm.

Results

The results obtained are summarized in Table I.

TABLE I
This table illustrates the kinetic parameters of the three abovementioned
substrates, compared with those obtained with the physiological substrates.

| | | Kₘ | K_{cat} | K_{cat}/Kₘ | Refer- |
|---|---|---|---|---|---|
| Enzyme | Molecule | [μM] | [sec-1] | [M⁻¹ × sec⁻¹] | ence |
| Recombinant | TS₂ | 45 | 240 | 5.3 × 10⁶ | [1] |
| TcTR | DTNB | Not | suitable | as | [13] |
| | | | | substrate | |
| | 1 | 35 | 125 | 3.6 × 10⁶ | |
| | 2 | 300* | 125 | 4.2 × 10⁵ | |
| | 3 | Not | suitable | as | |
| | | | | substrate | |
| Native | E. coli Trx | 34 | 5.6 | 1.6 × 10⁵ | [22] |
| hTrxR (lung | | | | | |
| adeno- | | | | | |
| carcinoma | | | | | |
| cells) | | | | | |
| Native | E. coli Trx | 20 | nd | nd | [29] |
| hTrxR | HTrx | 4.3 | nd | nd | [29] |
| (placenta) | DTNB | 400 | 67 | 1.7 × 10⁵ | [38] |
| | 1 | 7 | 46 | 6.6 × 10⁶ | |
| | 2 | 14 | 43 | 3.1 × 10⁶ | |
| | 3 | 10 | 38 | 3.8 × 10⁶ | |
| Recombinant | PfTrx | Physiological | | substrate | |
| PfTrxR | | not | yet | identified | |
| | E. coli Trx | 66 | >5 | nd | [26] |
| | DTNB | 1090 | 7 | 6.4 × 10³ | [26] |
| | 1 | 46 | 7 | 1.5 × 10⁵ | |
| | 2 | 400 | 17 | 4.3 × 10⁴ | |
| | 3 | 80* | 6 | 7.5 × 10⁴ | |

All the tests were carried out at 25° C. in 20 mM HEPES, 1 Mm EDTA, 150 mM KCl, at pH 7.25 (buffer A) and in the presence of 0.2 mM NADPH. In the tests marked with *, the reactions catalysed were accompanied by inhibition effects; thus, Kₘ and all the other values deduced were only able to be estimated at low substrate concentrations (15-200 μM).

The comparison of the parameters obtained when compound 1 or trypanothione disulphide is used as substrate for TR makes it possible to observe similar dynamic specificities, expressed as the K_{cat}/Kₘ ratio; they result from a lower Kₘ value (reduction of 30%), compensated for by a lower K_{cat} (reduction of 50%). As regards hTrxR, the largest increase in relation to DTNB as substrate consists in markedly reduced Kₘ values for disulphides 1 to 3. For example, FIG. 2 shows the reduction of compound 1 with the hTrxR enzyme: V max increases slightly, on the other hand with 7 μM, the Kₘ is much lower than that for DTNB (about 365 μM; ref. 29). Compound 2 according to the present invention and compounds 1 and 3 also have advantages as substrates for PfTrxR: the Kₘ of DTNB (about 1 mM; ref. 26) is even higher with PfTrxR than with hTrxR. Since it is difficult to carry out a test with more than 3 mM DTNB for problems of solubility, the measurements of specific activity of PfTrxR with DTNB can only be carried out with about 3×Kₘ. The use of disulphides 1 to 3, in particular of compound 1 (Kₘ=46 μM, Table I), thus contributes to solving this problem, by virtue of the much lower Kₘ values of these compounds.

The best affinity of the three disulphides (compounds 1, 2 and 3) in comparison with DTNB towards the NAD(P)H-dependent disulphide reductase enzymes probably comes either from the interactions of the ionic type between the protonated side amine-containing chains at physiological pH and the acidic residues of the active sites of the enzymes, or from the interactions of the cation/π type between the protonated side amine-containing chains and the aromatic residues of the active sites of the enzymes (D. A. Dougherty, Science, 1996, 271, 163-168; N. S. Scrutton et al., Biochem. J., 1996, 319, 1-8).

### EXAMPLE 3

### Protocol for the Screening of Inhibitors on Microtitre Plates

Materials and Methods

All the enzymatic and nonenzymatic reactions are carried out on Nunc plates (96 flat-bottomed wells) in a total volume of 100 μl. All the reaction media are incubated for 40 minutes at room temperature (22-25° C.) and stopped by the addition of 20 μl of acetonitrile.

The plates are then read with a Labsystems plate reader comprising a filter at 405 nm (Multiskan RC microplate reader Labsystems type 351, associated with a plate reader software Delta Soft III Biometallics (PRINCETON, N.J.).

For the single-point enzyme inhibition test (that is to say using only one concentration of the test compound), the following compounds are added to each well: 10 μl of solution at 500 μM of inhibitor in H₂O—Me₂SO at 10% (final concentration of 50 μM) and 10 μl of a solution of substrate in buffer A—Me₂SO at 10% containing 30 nmol of compound 1 and 50 nmol of NADPH (final concentrations of 300 μM for disulphide 1 and of 500 μM for NADPH). The reaction is triggered by the addition of 80 μl of an enzymatic solution containing 28×10⁻⁴ U of TcTR.

Suitable negative and positive controls are prepared in duplicate for each microtitre plate by incubating the following compounds during the entire duration of the test: a solution of substrate (final concentration of 2% of Me₂SO) with or without enzyme, a solution of substrate in the presence of enzyme and of reference inhibitor of TR, clomipramine (final concentrations of 50 μM of inhibitor, 2% of Me₂SO).

These conditions were also applied to the TrxR tests, with the following few modifications. To reduce the competitive reverse reaction of the oxidation of the thiolate formed, all the reactions with the TrxRs are carried out in buffer C (100 mM sodium phosphate, 2 mM EDTA, pH 7.0).

The final concentration of compound 1, 2 or 3 is 200 μM (instead of 300 μM in the TR tests). The reaction is triggered by adding 80 μl of an enzymatic solution containing either 8×10⁻⁴ U of native hTrxR, or 32×10⁻⁴ U of recombinant PfTrxR. The reference inhibitor of TrxR used in a positive control is 2,6-dichloroindophenol (final concentration of 25 μM in the hTrxR test and 50 μM in the PfTrxR test) [31].

Results

1) A first inhibition study consisted in monitoring the formation of the 5-thio-2-nitrobenzamide released by the reduction of compound 1 by measuring the absorbance at 416 nm and by monitoring it as a function of time, in the absence and in the presence of 50 μM clomipramine (FIG. 3).

On the other hand, the activity of TR in the presence of clomipramine (0-40 μM) was determined using either 5,5′-dithiobis(2-nitrobenzamide) 1 (20-200 μM), or T(S)₂ (trypanothione disulphide) (38), as substrate. The inhibition constants for clomipramine were deduced from the Lineweaver-Burk curves comprising on the y-axis the 1/V values and on the x-axis the 1/[S] values and corresponding curves representing the apparent Kₘ against [I]. In the presence of compound 1 as alternative substrate, clomipramine demonstrated, as expected, an inhibition of the competitive type with a Ki of 8.0±0.9 μM (FIG. 4b). This result is coherent with the value determined above in the presence of T(S)₂ (about 6.53±0.59 μM; [38]).

2) The activity of TR is then measured in the presence of compound 1 (50 μM) and of increasing concentrations of clomipramine (0-50 μM), by following the standard protocol for tests on microtitre plates as described above (FIG. 5). In this experiment as well, the results are consistent with the IC₅₀ values previously obtained by spectrophotometric methods (13). Thus, the novel colorimetric method according to the invention makes it possible to obtain a precise measurement of the TR activity and of the sensitivity of the inhibitor. The costs of this test are very low compared with the tests using T(S)₂ or analogues of T(S)₂ as substrate (supplied by Bachem, 100 mg of T(S)₂ costs about 1300 dollars). As regards the thioredoxin reductases, the superiority of the test according to the present invention, compared with the DTNB test, results from the increase in the affinity of the substrate, which makes it possible to work at low enzyme and substrate concentrations. This also allows appropriate solubilization of the inhibitors and reduces the probability of interference with other compounds in the test. For the three enzymes, the test developed can be carried out on microtitre plates, can be automated and is thus particularly suitable for a screening of inhibitors at high yield.

References

1. A. H. Fairlamb et al., Annu. Rev. Microbiol., 1992, 46, 695-729;

2. R. H. Schirmer et al., Angew. Chem. Int. Ed. Engl., 1995, 34, 141-154;

3. R. N. Ondarza et al., Arch. Med. Res., 1997, 28 (suppl.), S73-S75;

4. G. B. Henderson, J. Chem. Soc. Perkin Trans., 1990, 1, 911-914;

5. V. Fauchet et al., Bioorg. Med. Chem. Lett., 1994, 4, 2559-2562;

6. B. Kellam et al., Tetrahedron Lett., 1997, 38, 4849-4852;

7. I. R. Marsh et al., Tetrahedron, 1997, 53, 17317-17334;

8. A. El-Waer et al., Anal. Biochem., 1991, 198, 212-216;

9. A. F. El-Waer et al., Int. J. Peptide Protein Res., 1993, 41, 141-146;

10. R. Jaouhari et al., Amino Acids, 1995, 9, 327-342;

11. R. Jaouhari et al., Amino Acids, 1995, 9, 343-351;

12. I. R. Marsh et al., Eur. J. Biochem., 1997, 243, 690-694;

13. M. Aumercier et al., Anal. Biochem., 1994, 223, 161-164;

14. C. H. Jr. Williams, Chemistry and Biochemistry of Flavoenzymes, 1992, vol. III (Müller F. ed.) 121-211, CRC Press, Boca Raton;

15. A. Holmgren et al., Meth. Enzymol., 1995, 252, 199-208;

16. V. N. Gladyshev et al., Proc. Natl. Acad. Sci. USA, 1996, 93, 6146-6151;

17. N. Wakasugi et al., Proc. Natl. Acad. Sci. USA, 1990, 87, 8282-8286;

18. K. U. Schallreuter et al., Arch. Dermatol., 1987, 123, 1494-1498;

19. I. Saito et al., Arthritis & Rheumatism, 1996, 39, 773-782;

20. J. Kuriyan et al., Nature, 1991, 352, 172-174;

21. P. Y. Gasdaska et al., FEBS Lett., 1995, 373, 5-9;

22. T. Tamura et al., Proc. Natl. Acad. Sci. USA, 1996, 93, 1006-1011;

23. L. D. Arscott et al., Proc. Natl. Acad. Sci. USA, 1997, 94, 3621-3626;

24. S. Gromer et al., FEBS Letters, 1997, 412, 318-320;

25. S. Müller et al., Mol. Biochem. Parasitol., 1995, 74, 11-18;

26. S. Müller et al., Mol. Biochem. Parasitol., 1996, 80, 215-219;

27. K. Becker et al., Biochem. Soc. Trans., 1996, 24, 67-72;

28. R. L. Krauth-Siegel et al., Eur. J. Biochem., 1987, 164, 123-128;

29. J. E. Oblong et al., Biochemistry, 1993, 32, 7271-7277;

30. A. Nordhoff et al., Biochemistry, 1993, 29, 4022-4030;

31. B. L. Mau et al., Biochem. Pharmacol., 1992, 43, 1613-1620;

32. R. Fernandez-Gomez et al., Int. J. Antimicrob. Agents, 1995, 6, 111-118;

33. S. Baillet et al., C. Bioorg. Med. Chem., 1996, 4, 891-899;

34. S. Girault et al., Eur. J. Med. Chem., 1997, 32, 39-52;

35. B. Bonnet et al., Bioorg. Med. Chem., 1997, 5, 1249-1256;

36. L. Salmon et al., Chem. Pharm. Bull., 1998 (in press)

37. C. H. Faerman et al., Bioorg. Med. Chem., 1996, 4, 1247-1253;

38. T. J. Benson et al., Biochem. J., 1992, 286, 9-11.

As is evident from the above, the invention is not at all limited to those of its embodiments, implementations and applications which have just been described more explicitly; it encompasses on the contrary all the variants which may occur to the specialist in this field, without departing from the framework or from the scope of the present invention.

1. Substituted dithiobisnitrobenzenes, characterized in that they have the following general formula I: 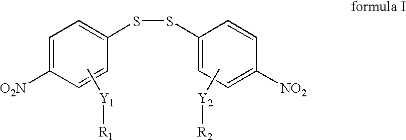
in which:
R₁ and R₂, which may be identical or different, represent:
a group NR₃R₄, in which R₃ and R₄, which may be identical or different, represent a hydrogen atom or a linear, branched or cyclic C₁-C₂₀ carbon chain comprising 1, 2 or 3 protonable nitrogen atoms,
a group OR₅, in which R₅ represents a hydrogen atom or an optionally substituted, linear, branched or cyclic C₁-C₂₀ carbon chain comprising 1, 2 or 3 protonable nitrogen atoms, on the condition that R₁ or R₂ represents a group NR₃R₄, in which R₃ and R₄, which may be identical or different, represent a hydrogen atom or a linear, branched or cyclic C₁-C₂₀ carbon chain comprising 1, 2 or 3 protonable nitrogen atoms,
an optionally substituted, linear, branched or cyclic C₁-C₂₀ carbon chain comprising 1 to 4 protonable nitrogen atoms,
or alternatively R₁ and R₂ are covalently linked, to form a macroring by formation of a chain of the NH—R₆—NH type in which R₆ represents a C₁-C₂₀ carbon chain comprising 1 to 4 protonable nitrogen atoms,
Y₁ and Y₂, which may be identical or different, represent a CH₂ group or a CO group, on the condition that when Y₁ and/or Y₂ represent a CH₂ group, R₁ and R₂, which may be identical or different, represent solely a group NR₃R₄, in which R₃ and R₄, which may be identical or different, represent a hydrogen atom or a linear, branched or cyclic C₁-C₂₀ carbon chain comprising 1, 2 or 3 protonable nitrogen atoms,
and on the condition that, when Y₁ and Y₂ represent a CO group, R₁ and R₂ do not simultaneously represent either a group —NH—(CH₂)₃—N(CH₃)₂ or a group:

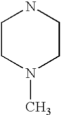

2. Substituted dithiobisnitrobenzenes according to claim 1, characterized in that Y₁ and Y₂ are identical and represent a CO group, the said derivatives corresponding to dithiobisnitrobenzamides and corresponding to the following formula II: 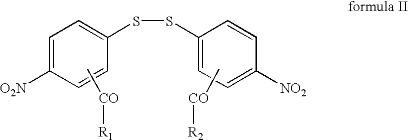
in which R₁ and R₂ are identical and represent a group NR₃R₄, in which R₃ and R₄ are different, one representing a hydrogen atom and the other representing a group 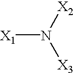
in which the groups X₁, X₂ and X₃ represent an optionally substituted, linear, branched or cyclic C₁-C₂₀ carbon chain comprising 1 to 4 protonable nitrogen atoms, an alkylamine group or an arylamine group, which are optionally substituted or branched, or alternatively X₁ and X₂ form a C₄-C₈ cyclic carbon chain which may optionally contain at least one nitrogen atom and X₃ represents a C₁-C₈ alkyl group, an alkylamine group or an arylamine group, which are optionally substituted or branched, the said group 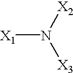
being linked by a carbon atom of at least one of the groups X₁, X₂ or X₃ to the nitrogen atom of the group NR₃R₄.

3. Substituted dithiobisnitrobenzenes according to claim 2, corresponding to dithiobisnitrobenzamides, characterized in that R₃ or R₄ represents a group:

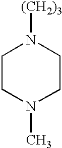

4. Substituted dithiobisnitrobenzenes according to claim 1, corresponding to dithiobisnitrobenzamides, characterized in that R₁ and R₂ are different, one representing a group OR₅ and the other representing a group NR₃R₄ as defined in claim 1.

5. Substituted dithiobisnitrobenzenes according to claim 1, characterized in that Y₁ and Y₂ are identical and represent a CO group and in that R₁ and R₂ form a chain NH—R₆—NH, in which R₆ represents a C₁-C₂₀ carbon chain comprising 1 to 4 protonable nitrogen atoms, the said derivatives corresponding to dithiobisnitrobenzamides.

6. Substituted dithiobisnitrobenzenes according to claim 1, characterized in that Y₁ and Y₂ are identical and represent a CO group and in that R₁ and R₂, which may be identical or different, represent an optionally substituted, linear, branched or cyclic C₁-C₂₀ carbon chain comprising 1, 2, 3 or 4 protonable nitrogen atoms, the said derivatives corresponding to dithiobisnitrophenacyls.

7. Substituted dithiobisnitrobenzenes according to claim 1, characterized in that Y₁ and Y₂ are identical and represent a CH₂ group, the said derivatives corresponding to dithiobisnitrobenzylamines and corresponding to the following formula III: 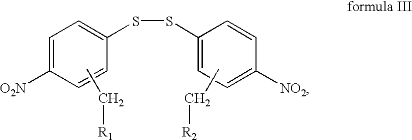
in which R₁ and R₂, which may be identical or different, represent a group NR₃R₄ in which R₃ and R₄, which may be identical or different, represent a hydrogen atom or an optionally substituted, linear, branched or cyclic C₁-C₂₀ carbon chain comprising 1, 2 or 3 protonable nitrogen atoms.

8. Method of preparing dithiobisnitrobenzamides according to any one of claims 2, 3 and 5, characterized in that it comprises:
(1) the conversion of 5,5′-dithiobis(2-nitrobenzoic) acid or its isomer 6,6′-dithiobis(3-nitrobenzoic) acid to an acylated derivative, in the presence of at least one coupling agent,
(2) the formation of an amide, from the product obtained in (1), by the action of an amine in excess and
(3) the purification of the product obtained in (2).

9. Method of preparation according to claim 8, characterized in that step (1) is carried out in the presence of a base.

10. Method of preparing the dithiobisnitrophenacyls according to claim 6, characterized in that it comprises:
(1) the formation of a diazoketone, from 5-chloro-2-nitrobenzoic or 2-chloro-5-nitrobenzoic acid,
(2) the hydrolysis of the diazoketone with hydrobromic acid,
(3) the substitution of the α-bromoketone with an amine and
(4) the formation of the disulphide, from the compound obtained in (3), by the action of Na₂S, S₈, in alcoholic medium, in accordance with scheme 1 below:

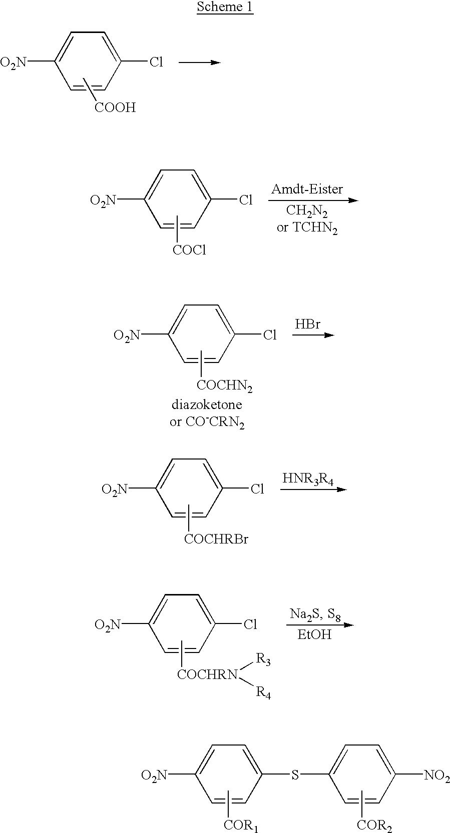

11. Method of preparing the dithiobisnitrobenzylamines according to claim 7, characterized in that it comprises:
(1) the reductive amination of an aldehyde 5-chloro-2-nitrobenzaldehyde or 2-chloro-5-nitrobenzaldehyde;
(2) the formation of the disulphide, from the compound obtained in (1), by the action of Na₂S, S₈, in alcoholic medium, under the same conditions as those stated in claim 10, in accordance with scheme 2 below:

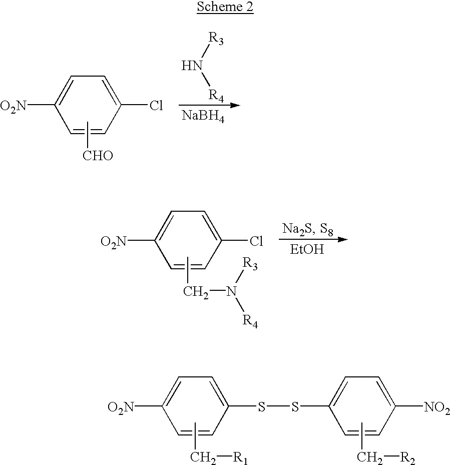

12. Method of preparing the dithiobisnitrobenzylamines according to claim 7, characterized in that the said dithiobisnitrobenzylamines are prepared from the following thiophenol: 2-mercapto-5-nitrobenzaldehyde, of the following formula: 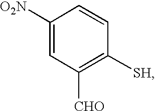
by the same reductive amination reaction as that described in claim 11, after protection of the thiophenol.

13. Method of preparing the dithiobisnitrobenzamides according to claim 4, characterized in that it comprises the following steps:
(1) the conversion of 5,5′-dithiobis(2-nitrobenzoic) acid to a monoester, according to a self-catalysed Fisher esterification reaction, with the aid of an alcohol of general formula R₅OH, R₅ being as defined in claim 1,
(2) the separation of the monoester obtained in (1) from the 5,5′-dithiobis(2-nitrobenzoic) acid and the diester also formed in (1), by chromatography, and
(3) the formation of an amide at the level of the monoacid function of the compound isolated in (2), by the action of an amine of general formula NHR₃R₄ in excess, R₃ and R₄ being as defined in claim 1, in the presence of at least one coupling agent.

14. Method of measuring the activity of the NAD(P)H-dependent disulphide reductase enzymes, whose active site comprises negatively charged amino acids and aromatic amino acids, capable of developing interactions with their substrate, either of the ionic type, or of the cation-Π type, which method is characterized in that it comprises:
bringing the said enzymes into contact with a suitable substrate selected from the group consisting of the substituted dithiobisnitrobenzenes according to any one of claims 1 to 7 and the substituted dithiobisnitrobenzenes of formula I as represented in claim 1, in which Y₁ and Y₂ represent a CO group and R₁ and R₂ represent simultaneously a group —NH—(CH₂)₃—N(CH₃)₂ or a group: 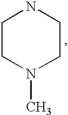
and
the direct detection of the thiolates formed.

15. Method of screening and selecting products inhibiting an NAD(P)H-dependent disulphide reductase, whose active site comprises negatively charged amino acid residues and aromatic amino acid residues, capable of developing interactions with their substrate, either of the ionic type, or of the cation-Π type, characterized in that it comprises:
bringing a potential inhibitor into contact, in the presence of an NAD(P)H-dependent disulphide reductase, with a compound selected from the group consisting of substituted dithiobisnitrobenzenes according to any one of claims 1 to 7 and substituted dithiobisnitrobenzenes of formula I as represented in claim 1 in which Y₁ and Y₂ represent a CO group and R₁ and R₂ represent simultaneously a group —NH—(CH₂)₃—N(CH₃)₂ or a group: 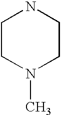
and
the colorimetric detection of the thiolates which may be formed.

16. Box or kit for measuring the activity of an NAD(P)H-dependent disulphide reductase, characterized in that it comprises, as alternative substrate for the said enzymes, a substituted dithiobisnitrobenzene in that it comprises, as alternative substrate for the said enzymes, a substituted dithiobisnitrobenzene selected from the group consisting of the substituted dithiobisnitrobenzenes according to any one of claims 1 to 7 and the substituted dithiobisnitrobenzenes of formula I as represented in claim 1 in which Y₁ and Y₂ represent a CO group and R₁ and R₂ represent simultaneously a group —NH—(CH₂)₃—N(CH₃)₂ or a group:

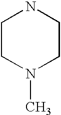

17. Method of assaying the NAD(P)H-dependent disulphide reductases, in a biological sample, characterized in that it comprises:
bringing the said biological sample, optionally treated, into contact with a suitable substrate selected from the group consisting of the substituted dithiobisnitrobenzenes according to any one of claims 1 to 7 and the substituted dithiobisnitrobenzenes of formula I as represented in claim 1, in which Y₁ and Y₂ represent a CO group and R₁ and R₂ represent simultaneously a group —NH—(CH₂)₃—N(CH₃)₂ or a group: 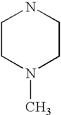
and
the direct detection of the thiolates formed, in particular by visible spectrophotometry.

18. Use of the method of detection according to claim 15, for the diagnosis of pathologies where the activity of NAD(P)H-dependent disulphide reductases is significantly increased.

19. Method according to any one of claims 14, 15 or 17, characterized in that the said NAD(P)H-dependent disulphide reductases are selected from the group consisting of trypanothione reductase, thioredoxin reductase and lipoamide dehydrogenase.
